(19) United States
(12) Patent Application Publication
(10) Pub. No.: US 2012/0088966 A1
(43) Pub. Date: Apr. 12, 2012

(54) Medical Nanobody

(76) Inventor: Clifford H. Kraft, Naperville, IL (US)

(21) Appl. No.: 13/073,019

(22) Filed: Mar. 28, 2011

Related U.S. Application Data
(63) Continuation of application No. 11/809,877, filed on Jun. 1, 2007, now abandoned.

(51) Int. Cl. A61B 1/00

(52) U.S. Cl. 600/104; 600/117

(57) ABSTRACT

A particle-sized nanobody that can be inserted into at least one major physiological system of a mammal's body such as the blood stream or the gastro-intestinal track or other system. The nanobody of the present invention can remain in the system for a predetermined time to perform a predetermined task. nanobodies of the present invention can contain processors and memory and thus can be capable of performing tasks that require algorithmic or expert reasoning. The Nanobodies can also contain various sensors and can optionally have the ability to communicate with an external station or with each other. The Nanobodies can be designed to self-destruct either after a predetermined time or upon command from an external station. Once a nanobody has self-destructed, natural mechanisms of the body can remove the debris.

Continuation of Ser. No. 11/809,877 filed Jun. 1, 2007. Ser. No. 11/809,877 is incorporated by reference.

### BACKGROUND

1. Field of the Invention

The present invention relates generally to a nonoscopic bodies that are injected into humans for medical purposes and more particularly to an internal human nanobody that can process and/or communicate.

2. Description of the Prior Art

It is known in the art to produce nano-bodies and to inject them into biological systems. Health Technologies February 2005 describes using metal nano-particles injected into a mammal's blood stream to cluster in tumor tissue and provide x-ray or other scanning reflectance so that the tumor shows up on CT-scans or other types of scans. Such metal particles (typically ferric oxide) can be maneuvered or directed by externally applied magnetic fields. (“Mit intelligenten Nanopartikeln gegen Krebs” (With Intellegent Nanoparticles against Cancer), Health Technologies, DGBMT, February 2005).

It is also known to coat particles with particular biological molecules such as proteins, enzymes, antibodies, etc. for identification or detection purposes (“Long-Circulating and Target-Specific Nanoparticles: Theory to Practice”, S. Moghimi et al., Pharmacological Reviews, June 2001).

The prior art does not teach a nanobody that contains a processor, and hence local intelligence, nor does the prior art teach a nanobody that can communicate with external sensors or other nanobodies and/or power itself from bodily fluids. It would therefore be advantageous to have a nanobody that could be injected into a mammal such as a human to perform diagnostics and/or treatment and which contains local intelligence or the ability to make decisions and/or the ability to communicate by either transmitting data, receiving instructions or communicating with other nanobodies, and optionally power itself from bodily fluids.

3. Description of the General Problem Solved by the Present Invention

The present invention relates to a nanobody with a processor and optional communication capabilities that is injected or entered into a human or animal body.

A nanobody can be injected or placed into various biological systems, organs and channels in a mammal such as a human. Among these are the circulatory system, the lymph system, the gastrointestinal system, the urinary system, the endocrine system, the brain, the heart, the kidneys, the liver, the spleen, the pancreas, the gull bladder, the bladder and in other systems or organs. Each of these systems has particular chemical and physical properties as well as certain channel sizes. For example, the circulatory system contains capillaries as small as 2 um in diameter, has a pH of around 7 and contains numerous types of cells, particles, proteins and other molecules. The gastrointestinal system, on the other hand, is characterized by large spaces and cavities, a pH which is acid in the stomach and alkaline in parts of the intestine.

A nanobody injected into a mammal may face attack by the immune system as a foreign body. For example, in the blood stream, a nanobody may be attacked by T-Cells, macrophages and other components of the immune system. Also, the injection of a large number of nanobodies might trigger a massive adverse immune response which could prove very dangerous for the patient.

Current micro-electronic processing technology can use line widths as small as 0.2 micron (um). This allows the production of a large processor (such as the Intel Pentium V) to be produced on a die of 5 mm on a side. Less complex processors and controllers can currently be produced on much smaller dies. Due to the shrinking size of transistors and line widths, moderately complex processors and controllers will soon be produced on dies as small as 10 microns or smaller. The technology to accomplish this currently exists with x-ray lithography and new revolutionary switching element designs. The present invention envisions nanobodies containing processors that range in size from around 1.5 micron to 40 or 50 microns on a side. Such nanobodies could be spherical or ellipsoidal shaped or could be flat with legs or cilia flagella or other propulsion means. Such nanobodies could emulate the shapes and propulsion techniques used by bacteria or use nano-motors with propellers or any other propulsion technique.

Nanobodies that communicate with external sensors or with each other need miniature communication circuits and techniques that could range in sophistication from simple RFID functions to full-fledged full-duplex data communications. Data communications would have to take place in the particular channel or environment that the nanobody was being used in and would exploit the use of some type of carrier energy such as radio, light, sound, magnetic fields or any other way of transmitting intelligence from one point to another.

A nanobody containing a processor and/or communications capability as well as propulsion needs and energy source. The simplest could be a nano-battery that was either self-contained or operated out of a portion of an electrolyte found in the channel (such as blood serum). A considerably more sophisticated energy source might be the actual metabolism of biological products from the channel itself (such as the metabolism of glucose from blood serum). Such metabolism could simply mimic some of the pathways used by the organism or by known bacteria; however, these are generally quite complex chemically requiring the presence of numerous enzymes and sophisticated support in terms of membranes, etc. to make sure each step in the pathway is spatially and chemically separated for other steps. However, it is not necessary that such a metabolism follow the same chemical and physical steps used by any particular organism. As will be explained, shortcut chemical paths exist that can produce electrical energy from metabolic products like glucose with less steps than are found in natural organisms.

The end product of a nanobody power source would be a continuous flow of enough electrical current in a correct voltage range to power a processor and/or communications circuits and/or nano-propulsion. A nano-power source would most likely pump electrical charge into a capacitor or mini-battery until it reached a desired voltage. A type of regulator could keep the voltage constant while current was drawn out of the capacitor. A battery would maintain a constant voltage. The capacitance or battery would have to be adequate to supply the current needed by the load. Thus, a particular embodiment of a nanobody power supply could contain a charge pump powered by chemical energy, a capacitor and a regulator. As stated before, a self-contained rechargeable or non-rechargeable nano-battery could be used to supply all of these roles.

Injecting or placing nanobodies into a human or animal would generally be done with a particular purpose in mind. While such systems can find many uses, there are numerous possible purposes; two primary ones would be: 1) measuring, sensing or evaluating, and 2) actively combating disease or tumors. It is currently known to have a patient swallow a tiny camera that then finds its way into the intestinal track. This camera can send video or images to an external receiver. This is an example of the first group of applications. This prior art camera does not contain any intelligence except what is necessary to gather and transmit an image. An example of the second type of applications would be a nanobody that could bring about or participate in the killing of a bacterium, virus or tumor cell. The present invention is directed to both types of applications.

The following possible uses are envisioned for the nanobody of the present invention: 1) killing viruses, bacteria, or tumor cells; 2) repairing organs or cells; 3) removing pieces of clot or plaque; 4) taking place of a cell—acting as artificial cell for a specific purpose; 5) making specific measurements; 6) detecting unwanted cells or other material; and, 7) delivering drugs to specific sites. These are just examples of possible uses of the present invention. One of skill in the art will realize that there are numerous other uses that are within the scope of the present invention.

A final problem relating to and solved by the present invention is how nanobodies can be removed from the body once they have completed their tasks. The present invention envisions several ways: 1) self-destruction (dissolving); 2) eaten by macrophages or other body cells; 3) removed by spleen or kidney; 4) filtered mechanically (such as by dialysis filtering); 5) the nanobody remains indefinitely.

### SUMMARY OF THE INVENTION

The present invention relates to a particle-sized nanobody that can be inserted into at least one major physiological system of a mammal's body such as the blood stream or the gastro-intestinal track or other system. The nanobody of the present invention can remain in the system for a predetermined time to perform a predetermined task. nanobodies of the present invention can contain processors and memory and thus can be capable of performing tasks that require algorithmic or expert reasoning. Nanobodies of the present invention can also contain various sensors and can optionally have the ability to communicate with an external station or with each other. Nanobodies of the present invention can be designed to self-destruct either after a predetermined time or upon command from an external station. Once a nanobody has self-destructed, natural mechanisms of the body can remove the debris.

### DESCRIPTION OF THE FIGURES

FIG. 1 shows injection of nanobodies into a mammal.

FIG. 2 is a sectional diagram of a nanobody.

FIG. 3 shows an embodiment of a power source or power module for a nanobody.

FIGS. 4A-4B show possible use-modules for a nanobody.

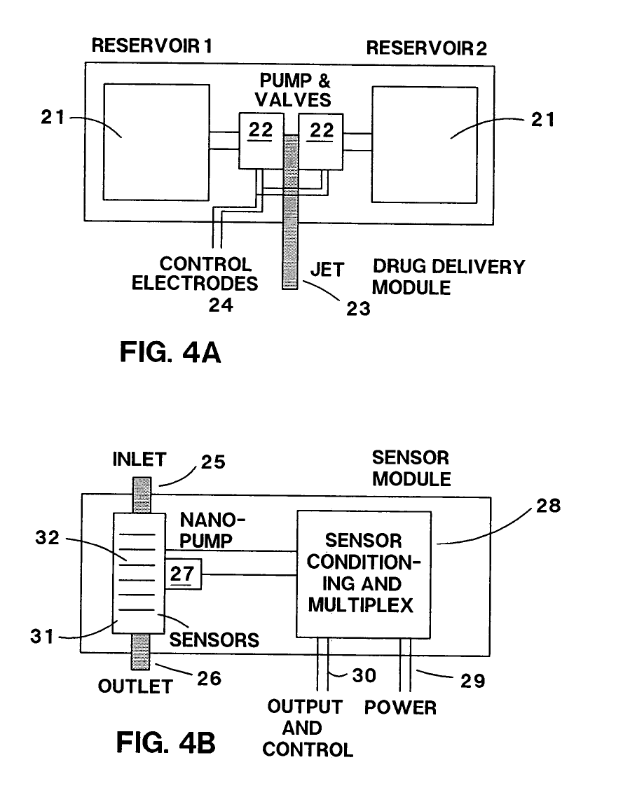

FIG. 5 shows a communications module for a nanobody.

FIGS. 6A-6B show an embodiment of a folding nanobody that can pass through very small capillaries.

Several drawings and illustrations have been presented to aid in the understanding of the present invention. The scope of the present invention is not limited to what is shown in the figures.

### DESCRIPTION OF THE INVENTION

The present invention relates to a nanobody or group of nanobodies that can be inserted into a major physiological system of a mammal, and in particular into a human being. An important feature of the invention is that some or all of the nanobodies can contain a processor and memory and can thus perform tasks that require artificial intelligence and/or algorithmic capability. In addition, nanobodies of the present invention can optionally have the capability of communication with either one or more outside stations or with each other.

The surface of the nanobody can be equipped with various sensors and also can be coated with proteins or other biological material to trick the immune system into believing that the nanobody is friendly or for other functions The processor part of the nanobody can generally be constructed from a semiconductor material, and other parts can be made of material that is more biologically compatible and even eventually biodegradable (polysaccharide polymers for example). Various biologically compatible materials could be designed to react in a particular way with the body environment (for example dissolving slowly). For flexibility and almost total water insolubility, the polysaccharide cellulose can be used (cellulose is a linear polymer of up to 3000 units of D-Glucose linked by beta-1,4-glycoside bonds. It is also possible to use protein or protein-like structures such as derivatives of beta-tubulin, G-actin, F-actin and myosin.

The nanobodies of the present invention can be designed to accomplish various tasks including making local measurements and checks, checking for tumor cells, actively attacking tumor cells, bacteria or viruses or helping to repair damaged tissue or structure. Nanobodies can be designed to remove plaque from blood vessels, to act as artificial T cells or other parts of the immune system (for example for AIDS patients) or for any other purpose.

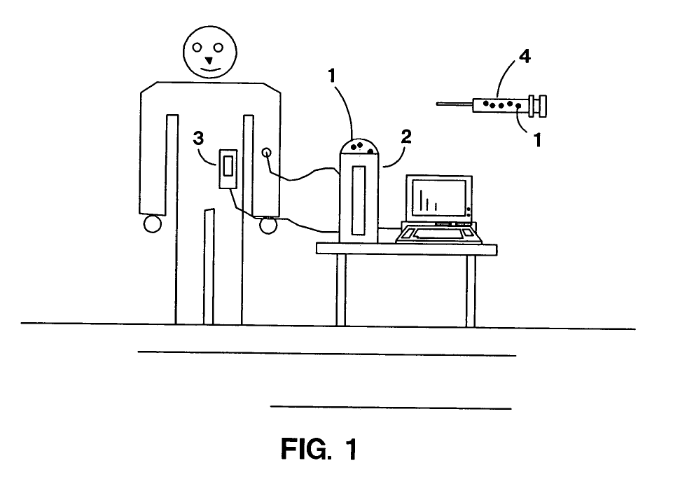

FIG. 1 shows a method of injecting nanobodies into a human along with a method of communicating with them. Nanobodies 1 can be injected into the blood stream by a standard syringe 4 or by using a micro-precision pump system 2 like that described by Bach et al. in U.S. Pat. No. 6,739,478. The advantage of a precision dispensing system is that the exact number of injected nanobodies can be controlled. In the particular embodiment shown in FIG. 1, communication with the nanobodies is through a magnetic field transmitter 3. This transmitter can modulate a DC or AC magnetic field with data that can be sensed using a micro-magnetometer in the nanobody. Optionally, the magnetic transmitter 3 can also contain a sensitive magnetic or RF receiver to receive signals from particular nanobodies. Each nanobody can be coded with a particular code to identify its communications from that of other nanobodies. RF signals can be received from nanobodies passing through capillaries near the skin surface and can be generated by very small oscillators. Magnetic signals can be generated by nanobodies deep in the body and received near the surface of the body. Particular communication schemes will be subsequently discussed.

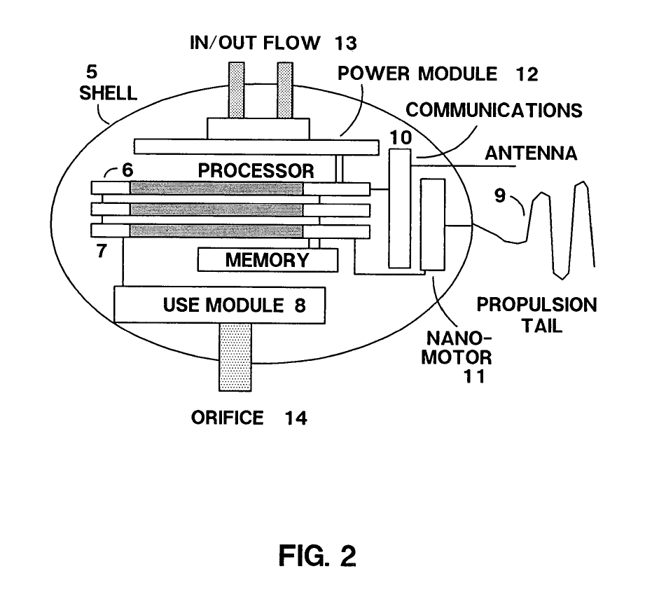

FIG. 2 shows a cross section of a nanobody. The entire nanobody is contained inside a shell 5. As has been stated, the shell 5 can optionally be made of a material like a polysaccharide that is bio-compatible or will eventually dissolve. A processor 6, normally with embedded memory, is attached to or made on a substrate 7. The processor can be layers of etched semi-conductor material like silicon or any other material including amorphous material. The processor can communicate with a use-module 8, a power module 12 and an optional communications module 10. A nanomotor 11 can be used to power a propulsion means 9 like a rotating tail. The power module can be a battery, or optionally can generate electricity from the surrounding fluid though the use of in- and out-flow channels 13. The use module 8 may have an optional orifice 14 to deliver drugs or take fluid samples.

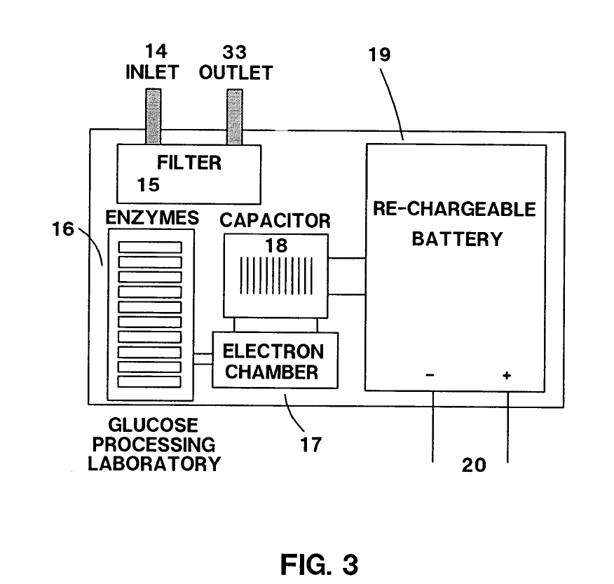

FIG. 3 shows an embodiment of a power module that runs on glucose from the blood. Blood serum enters an inlet orifice 14 and flows into a filter 15 that removes any particles or cells. The filter should remove any body larger than 1 micron. The filter can be a zeolite or other micro-structure filter. The filtered fluid enters a glucose processing laboratory 16 which contains a number of reaction cells containing enzymes. Each enzyme converts an input compound into an output compound. A final output compound enters an electron chamber 17 where a battery-like chemical reaction mimics the reaction that takes place in particular membrane proteins in mammal cell membranes that produce an electrical potential that causes electrons to flow into a capacitor 18 charging it (such as the process at the end of major glucose metabolism chains that takes place on the endoplasmic reticulum of cells where an ATPase converts a proton or electron current into ATP). The chemical process keeps the capacitor charged while it in turn charges a mini-rechargeable battery 19. The mini-battery 19 supplies the correct voltage across its terminals 20 to power the processor and all other circuitry in the nanobody. Unused serum can exit an outlet orifice 33.

The proceeding example of a power source is given to better aid in understanding how the nanobody of the present invention can be powered. As previously stated, the power source can be a simple, non-rechargeable battery, or any other power source including a miniature hydrogen fuel cell. Miniature fuel cells that derive hydrogen from alcohols are known in the art. Power can also be supplied from outside the body by RF, light or any other type of energy. A changing magnetic field can be used to produce an electromotive force (EMF) across a small conductive coil. Any type of power source for a nanobody is within the scope of the present invention.

The purpose of the nanobody can be one or more of many possible purposes. For example, the nanobody may be injected to deliver drugs or sense conditions such as temperature, sodium or potassium (or other ion) concentration, toxicity, glucose level, levels of other body chemicals or enzymes, etc. The purpose could also be tissue repair, cell or virus attack, plaque destruction, delivery of a micro-implant device or one of almost endless possibilities. Such purposes can be accomplished through use modules that could be put into nanobodies either when manufactured, or later when needed. FIGS. 3A-3B show embodiments of two use modules: a drug delivery module and a sensor module.

Turning to FIG. 3A, an embodiment of a drug delivery use module can be seen. This particular embodiment can deliver two different drugs if desired. A pair of reservoirs 21 contain liquid drug samples. Pumps and valves 22 control delivery through an exit jet 23 so that the drug can be delivered to an exact location. Control electrodes 24 can be driven by the processor to initiate and control the delivery.

FIG. 3B shows a sensor use module. An inlet 25 feeds through a sensor chamber 31 containing several sensors 32. A nano-pump 27 can cause liquid to enter the inlet 25, pass through the sensor chamber 31, and exit through an outlet 26. This circulation can be carried on continuously or can be started when needed to conserve power. A sensor signal conditioner 28 can condition the output put of each sensor, optionally convert it to digital format and optionally multiplex it on a signal output line 30 for sending data to the processor. A set of power leads 30 can supply power for the conditioner, sensors and nano-pump.

In a particular embodiment of the present invention, the processor can receive updated instructions, data or parameters from the communication module 10 (FIG. 2), which in turn can receive this data from a transmitter external from the body. Alternatively, the processor can receive messages from other nanobodies also in the mammal. In addition, the processor can optionally transmit data to an external receiver or to other nanobodies.

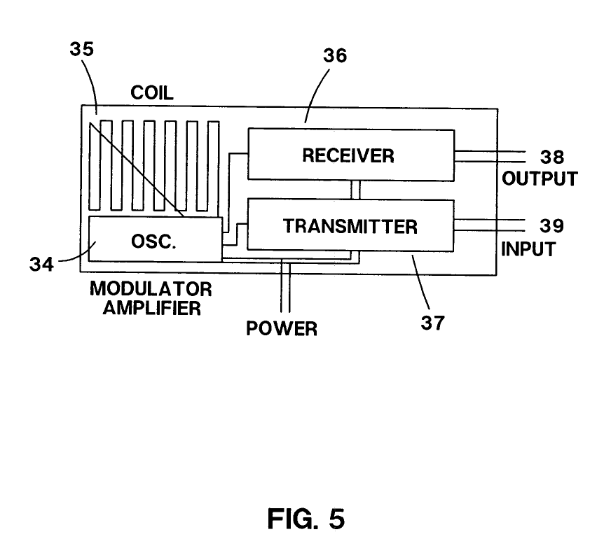

Turning to FIG. 5, an embodiment of a magnetic or RF communications module can be seen. A oscillator/modulator/amplifier 34 drives a coil or loop antenna 35. In receive mode, it amplifies incoming signals from the coil 35. Incoming signals can be sent to a receiver 36 that produces a digital output 38. In transmit mode, a transmitter 37 receives digital signals from an input 39 and causes the oscillator's output to be modulated at a particular data rate and fed through the coil or loop antenna 35. This causes either electromagnetic radiation if the frequency is high enough or simply a slowly changing magnetic field. Either case is really the same, since a loop antenna always produces a radiation near field that is primarily magnetic. Because of tremendous attenuation any AC field exiting the nanobody, the preferred method is to use an almost static magnetic field that is modulated at a very low data rate (such as less than 1 kHz). Any type of electromagnetic device operating at any frequency is within the scope of the present invention. Light, thermal, and other methods are also possible. For example, communication with one or only a few nanobodies is possible from modulating a laser directed into their area. The thermal changes could be sensed and used to read the data by the nanobody. Any type of communication to or from a nanobody is within the scope of the present invention.

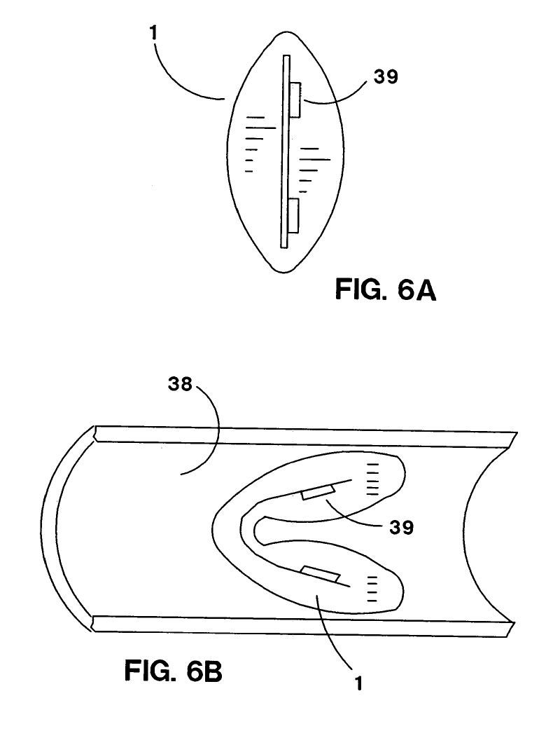

The size of the smallest capillary in the human body is around 2-5 micron in inner diameter, while the smallest venule is around 2 micron in inner diameter. A red blood cell (erythrocyte) is approximately 6-8 micron in diameter. The red blood cell passes through a capillary that is smaller than its diameter by distorting itself under the pressure of the blood flow and the capillary walls. FIG. 6A-6B show a nanobody 1 that mimics a red blood cell. The nanobody can be made of softly elastic material with islands of electronics 39 that can distort as shown in FIG. 6B when passing through a small capillary. Alternatively, the nanobody can be made as a frame that folds in the capillary. This type of folding procedure is only necessary for nanobodies that circulate in the blood stream. The nanobody of FIG. 6A 1 can have a diameter of from 1.0-1.5 micron provided it can distort enough to pass through a capillary of 2 micron in diameter. To accomplish the smooth transition into the distorted mode of FIG. 6B, it is generally necessary to coat the nanobody with a lubricant or mucus-like substance. A natural substance like that found on the cell surfaces of red blood cells can be used, or synthetic lubricants can be employed. Generally there is a boundary layer of blood serum between a red blood cell and the capillary wall; the same is true for a nanobody if it is designed to resemble a red blood cell. Any lubricating method is within the scope of the present invention. It is also helpful if bloodstream nanobodies carry correct blood identifier sugars (small polysaccharide chains) to be compatible with various blood types (such as type A, B, AB and O). It is well known that blood type A erythrocytes contain membrane surface tetra-saccharides made up of the following monosaccharides: NAGal-GAL-NAGal with Fuc branched from the center GAL. Blood type B is the same except the leading NAGal is replaced with GAL. Blood type O is totally missing the leading monosaccharide and is thus a tri-saccharide. Blood type AB has both A and B tetra-saccharides present.

While various uses and embodiments of a nanobody have been presented, nanobodies can perform many different tasks and functions in clinical medicine. Nanobodies can be disguised from the immune system or become part of it with the correct recognition proteins or sugars on their surfaces. Nanobodies can repair desirable tissue or destroy undesirable tissue (like tumors). With communications and processors, nanobodies are able to coordinate efforts where hundreds or even thousands of nanobodies coordinate a particular task. With communications and processors, separated nanobodies can find each other and nanobodies that are grouped can separate to perform diverse tasks. The present invention envisions numerous improvements in and reductions in size in processors, electronics, power sources, delivery systems, sensors and communications. All of these improvements and reductions in size are within the scope of the present invention.

Several examples, descriptions and illustrations have been presented to better aid in understanding the present invention. One skilled in the art will realize that there are many changes and variations that can be made without departing from the spirit of the invention. Each of these changes and variations is within the scope of the present invention. In addition, one skilled in the art will recognize that the technology continually changes. The present invention envisions numerous changes and improvements in the technology that relate to how the principles of the present invention will be implemented. The use of new technology and changes and improvements in older technology as they relate to the invention are within the scope of the present invention.

1. A medical nanobody adapted to enter a human body comprising:
a propulsion mechanism;
a shell containing a processor, a memory, a use module, a power module and a communication module, said processor in data communication with said memory, said memory containing executable instructions and data;
said processor executing computer instructions from said memory and reading and writing data from and to said memory, said processor connected to said use module, wherein said use module allows interaction with said human body, and said communication module allows 2-way communication with a station external to said human body or to another nanobody inside said human body.

2. The medical nanobody of claim 1 wherein said power module is an internal energy generation system.

3. The medical nanobody of claim 2 wherein said internal energy generation system is metabolic.

4. The medical nanobody of claim 1 wherein said power module is a battery.

5. The medical nanobody of claim 1 wherein said use module contains at least one sensor.

6. The medical nanobody of claim 27 wherein said use module is adapted to kill particular cells in said human body.
